(19) United States
(12) Patent Application Publication
(10) Pub. No.: US 2007/0185410 A1
(43) Pub. Date: Aug. 9, 2007

(54) Multiple collection kit for stool laboratory examination

(75) Inventor: Carlos Cesar Moyano Fernandes, Sao Paulo (BR)

Correspondence Address: DAVIDSON, DAVIDSON & KAPPEL, LLC, 485 SEVENTH AVENUE, 14TH FLOOR, NEW YORK, NY, 10018, US

(73) Assignee: Immunoassay Industrial E Comercio Ltda., Sao Paulo (BR)

(21) Appl. No.: 10/914,725

(22) Filed: Aug. 9, 2004

(51) Int. Cl. A61B 10/00; A61B 5/00; B01L 11/00

(52) U.S. Cl. 600/562; 600/573; 422/101

(57) ABSTRACT

“MULTIPLE COLLECTION KIT FOR STOOL LABORATORY EXAMINATION”, consisting of a set of vials (10) for the collection or storage of collected stool specimens and a specimen concentrator container (20), being each of the collection vials (10) equipped with its own specimen collection scoop (7) and a closing cap (6) adapted by clicking close to the access opening (3, 4) of the vial (10), with such opening dimensionally compatible with click coupling in one of the three corresponding inlet openings (25c) provided in the specimen concentrator container (20), which is internally equipped with a filtration set (30) positioned on a parallel and intermediate plan in relation to the entry openings of the specimens collected (25c) and to the storage chamber (21, 22) formed by the concentrator container (20), being such transfer performed by centrifugation.

This report refers to an invention patent for a multiple collection kit for stool examination and, in particular, to a number of parts forming the set of storage vials for the stool specimens, to be provided to the user, and a concentrator container for specimens collected, which is kept at the lab for use by technical personnel, to assemble the kit and process specimens collected.

As known by the professionals of the area, in order to perform a laboratory diagnosis, when the collection of more than one specimen is required, such as stool parasitologic examinations, the user should collect the stool¹ specimen on three alternate days and have them stored in a corresponding sterile pot available from drugstores or furnished by the laboratory itself.

Although this system of stool collection and delivery to the laboratory is a reasonably satisfactory practice, it poses some inconveniences, to the extent that it makes collection embarrassing to the user and specimen handling difficult to the laboratory personnel.

Another inconvenience found in this usual practice of stool collection for laboratory examination refers to the use of the traditionally adopted pots, which implies a high degree of contamination as a result of inappropriate handling at the time of stool collection, as well as at the time the specimens collected are transferred to the specimen concentrator container, which remains at the lab.

Still a third problem identified in this usual practice of stool collection refers to the form of the pots for specimen collection, which require that the specimen is stored with the use of specific tools, both by the user at the time of collection and by the lab technician for the transfer of specimens collected to an already standardized laboratory system to process them.

Examinations for the diagnosis of intestinal parasitosis usually involve handling of fecal specimen. More practical kits, however, are available in the market, with the capacity to process only one stool specimen, for instance, Coprotest, a trade name owned by NL Comércio Exterior Ltda. ³, the single kit currently marketed in the country, and others marketed only abroad, such as ParaSep, a trademark registered to DiaSys Co. ⁴, FPCJumbo, a trademark registered to Evergreen Scientific Co., ParaSed, trademark pertaining to Medical Chemical Co., and Macro-Com, a trademark owned by Meredian Diagnostic Inc. ².

One of the objectives of the invention at issue is, therefore, to provide a kit for multiple stool collections for parasitological examination, which renders significantly easier and more practical the routine of collecting and delivering stool specimens to the lab, i.e., without causing embarrassment to the user during collection or material handling difficulties to the lab personnel.

Another objective of this invention is to provide a kit for multiple collections of stool specimens for parasitological examination already equipped with a set of vials, each of them with its collecting scoop, which eliminates the likelihood of contamination for inadequate stool handling during collection, in addition to eliminating specimen handling at the time it is processed in a standardized laboratory system.

A third objective of this invention is to provide a kit for multiple collection of stool specimens for parasitological examination which does not require handling of the material stored therein with specific tools by the lab technician, as he/she transfers the specimens collected into the concentrator container system.

These and other objectives and advantages of the present invention are made possible through the use of a multiple stool collection kit for lab examination, which includes a set of three collection or storage vials to provide the user, as well as a specimen concentrator container. Each of the vials is equipped with its own stool collecting scoop and a click closing cap adapted by clicking to the access opening of the vial, which opening is dimensionally fit to be coupled by clicking to one of the three corresponding entry openings provided in the specimen concentrator container, which in turn is equipped inside with a set of filters positioned on a plan which is parallel and intermediate to the openings for inserting vials with specimens collected and the storage chamber represented by the concentrator container, being such transfer performed by centrifugation.

The invention will be described below with references to the drawings attached, where:

FIG. 1 represents a perspective view of one of the specimen collection vials, from the collection set to be sent to the user. This is a representation of the vial as closed.

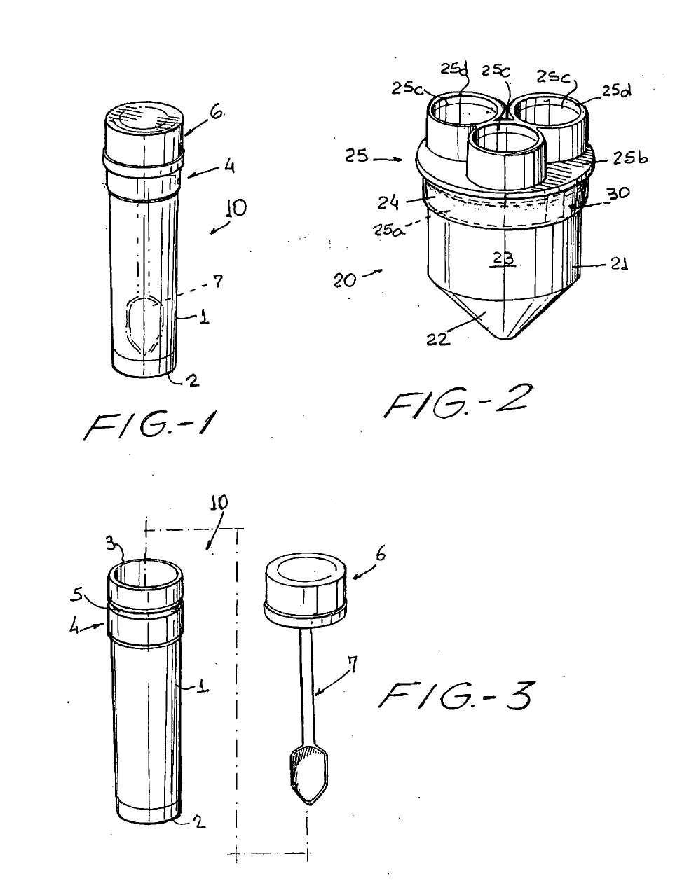

FIG. 2 represents a perspective view of the specimen concentrator container, in its assembled form and ready to receive the vials with stool specimens.

FIG. 3 represents a perspective view of one of the specimen collection vials open and ready to receive a stool specimen;

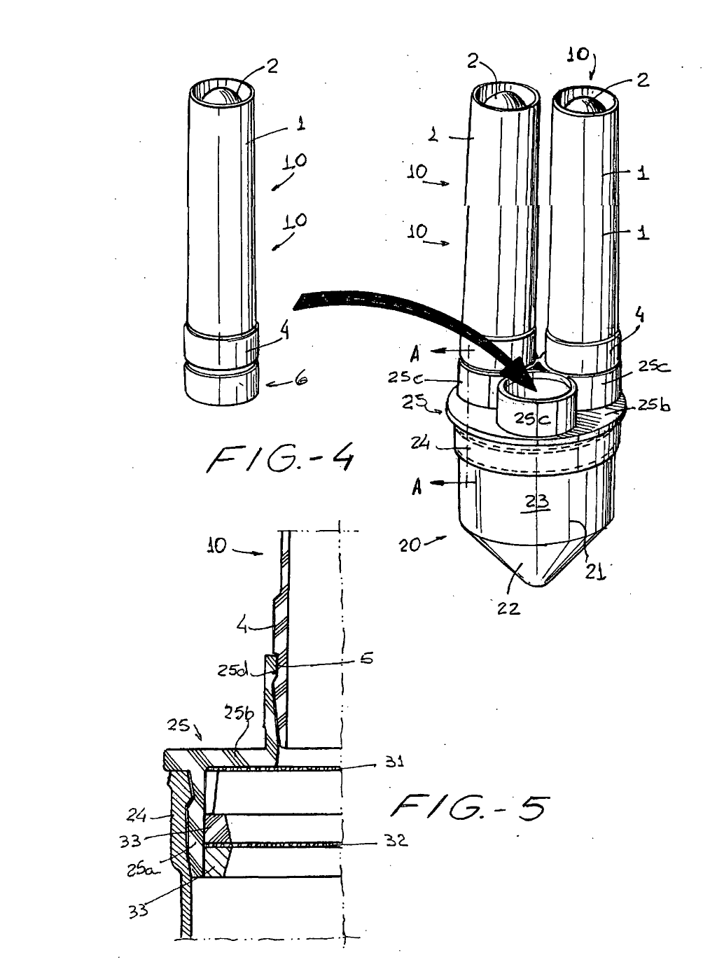

FIG. 4 represents a perspective view of the collection vials being coupled at the entries of the specimen concentrator container;

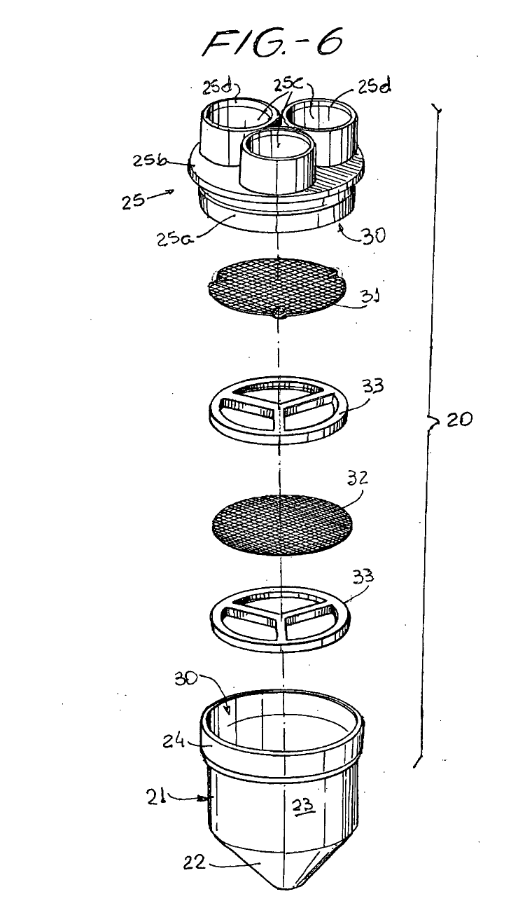

FIG. 5 represents an enlarged and section detail of the coupling between the vial and the container, taken according to the “A-A” line of FIG. 4; and

FIG. 6 represents perspective view of the specimen concentrator container as knocked-down, with drawings of all the container components.

According to such drawings, the multiple collection kit for stool examination consists of a set of three collection or storage vials (10) for stool specimens, to provided the user, and one specimen concentrator container (20), the first ones being selectively and adequately coupled to the second at the time of the simultaneous transfer of the specimens collected into a specimen aggregation chamber. See FIG. 4.

According to FIGS. 1 and 3, each of the collection vials (10) consists of a tubular body (1), with the bottom end closed (2) and the upper end open (3), which external peripheral border includes a radial thickening (4) with a perimeter groove (5), thus forming a border for precise fitting, click retention and a bung of an upper closing cap (6) of short tubular format and internal diameter slightly larger than the external diameter (3) of the vial (10) opening, so that it can be precisely attached in the axial direction to the latter.

As can be seen in FIG. 3, a specimen collecting scoop extends downwards, in an internal and axial projection in relation to the closing cap (6), in the form of a specimen collecting spoon or scoop (7), which has its own cap (6) as manual holder, thus allowing the user to collect a specimen of the stool to be examined and simultaneously store it in, and close the vial, as shown in FIG. 1. It is worth emphasizing that the collection vial (10) is already provided to the user with the preservative solution inside, and its direct contact with the specimen collected occurs only through the specimen collecting scoop (7), which is kept inside the vial (10) after closure.

As can be seen in FIGS. 2, 4, 5 e 6, the specimen concentrator container (20) consists of a substantially trunk-conic body (21), with its bottom part (22) in a closed conic form and a tubular (23), open upper part, which peripheral external border includes a diameter enlargement (24), forming the border for a precise axial and pressure-fitting of an upper closing cap (25) with a bottom part (25a) in a short tubular form and with its external diameter slightly smaller than the internal diameter (20) of the container opening (24), allowing the axial and precise fitting of such cap (25) with the container (20) opening (24). The upper part of this closing cap (25) is formed by a circular platform (25b) with a diameter larger than the one defined for its bottom part, so that the peripheral border of this platform (25b) acts as axial stop for the cap (25), fitting it on the enlarged circular border (24) of the container (20), as shown in FIG. 5. Also to platform (25b) of the cap (25) are incorporated three circular inlet openings, from which the corresponding short tubular projections (25c) extend upwards, with their internal diameter slightly larger than the external diameter of vial (10) opening (3), with a radial collar (25d) inside the open circular border of each of such tubular projections (25c), which enables the click retention of the vial (10) by one of the tubular projections (25c). Between the platform (25b) level and the lower border of the bottom part (25a) of such upper closing cap (25), there is a filtration chamber (30), consisting of, at least, two filtering levels, each of them with two screens (31/32) of different permeability, which are kept parallel to each other and immobilized inside the cap (25) bottom part (25a) through the ring supports (33), pressure-mounted and inserted alternately in relation to said screens (31 and 32) inside the cap (25) bottom part (25a), as shown in FIG. 6.

This way the user, having collected and inserted the three specimens in their respective vials (10), delivers them simultaneously to the lab, which, in turn, without any handling of the material collected, opens the vials and fits each of them to one of the tubular projections (25c) of the specimen concentrator container (20), in the case of this coupling laid upside down to avoid leakage of the specimens.

Upon coupling of the specimen vials (10) to the concentrator container (20), the set is submitted to centrifugation, in such a way that the specimen collected and previously dipped into the preservative solution contained in each vial (10) is thrust by centrifugal force to the inside of the trunk-conic body (21), with the closed lower conic part (22) of the container (20) going through the filtration chamber (30). Thus, the three stool specimens initially collected in different vials (10), after centrifugation, will be aggregated as a single amount of filtered material, forming a pellet to be examined, the transfer and aggregation occurring without any handling.

Despite having being described and illustrated as a preferred constructive design, it must be stressed that the kit at issue may present some structural alterations without, however, deviating from the scope of the present invention.

1. “MULTIPLE COLLECTION KIT FOR STOOL LABORATORY EXAMINATION”, characterized by a set of vials (10) for the collection or storage of the specimens collected and a container (20) for the specimen concentration, being each of the collection vials (10) provided with its own collecting scoop (7) and a closing cap (6) adapted by clicking and a bung by the access opening (3, 4) of the vial (10), such opening being dimensionally compatible with click coupling to one of the three corresponding inlet openings (25c) in the specimen concentrator container (20), which is internally equipped with a filtering set (30) positioned on a parallel and intermediate plan in relation to the openings for transfer of the specimens collected (25c) and to the storage chamber (21, 22) formed by the concentrator container (20), being the transfer performed by centrifugation.

2. “MULTIPLE COLLECTION KIT FOR STOOL LABORATORY EXAMINATION”, pursuant to claim 1, is characterized by the furnishing of three vials (10) for collection or storage of specimens collected, to be provided to the user, and a specimen concentrator container (20) for the specimens collected, the first one being selective and adequately coupled to the second at the time of the simultaneous transfer of the specimens collected into a specimen aggregation chamber.

3. “MULTIPLE COLLECTION KIT FOR STOOL LABORATORY EXAMINATION”, according to claim 1, is characterized by the collection vials (10) each of them formed by a tubular body (1) with the bottom end closed (2) and upper end (3) open, which peripheral border includes a radial thickening (4, 5) forming a border for precise fitting and click retention of an upper closing cap (6) in a short tubular form with an internal diameter slightly larger than the external opening diameter (3) of the vial (10), so that it can be precisely be fit to the latter.

4. “MULTIPLE COLLECTION KIT FOR STOOL LABORATORY EXAMINATION”, according to claim 1, is characterized by the furnishing of a specimen collecting scoop (7) extending downwards internally and axially to the closing cap (6), in the form of a spoon or scoop with its own cap (6) as the manual holder, thus allowing the user to collect the specimen of the material to be examined and simultaneously store it in, and close the vial.

5. “MULTIPLE COLLECTION KIT FOR STOOL LABORATORY EXAMINATION”, according to claim 1, is characterized by the specimen concentrator container (20) consisting of a substantially trunk-conic body (21) with its bottom part (22) in a closed conic form and a tubular and open upper part (23), which peripheral border includes a diameter enlargement (24), forming a border for precise axial fitting by pressure of an upper closing cap (25) and a bottom part (25a) in short tubular form and external diameter slightly smaller than the internal opening diameter (24) of the container (20), being the upper part of such closing cap (25) formed by a circular platform (25b) with a diameter larger than the one defined for its bottom part, so that the peripheral border of this platform (25b) acts as axial stop for the cap (25), fixing itself over the enlarged circular border (24) of the container (20); on platform (25b) are transfer circular openings, from which corresponding short tubular projections (25c) with internal diameter slightly larger than the external opening (3) diameter of the vial (10) extend upwards.

6. “MULTIPLE COLLECTION KIT FOR STOOL LABORATORY EXAMINATION”, pursuant to asseverations 1 to 5, is characterized by the furnishing of a filtration chamber (30) between the platform level (25b) and the lower border of the bottom part (25a) of said upper closing cap (25), which chamber consists of, at least, two filtering levels, each of them formed by screens (31) and (32) with distinct permeability, parallel to each other and maintained immobilized inside the bottom part (25a) of the cap (25) through the ring supports (33) pressure- mounted and positioned alternately in relation to said screens (31 amd 32) inside the bottom part (25a) of the cap (25).
